(19) United States
(12) Patent Application Publication
(10) Pub. No.: US 2002/0039422 A1
(43) Pub. Date: Apr. 4, 2002

(54) Driving mode for active noise cancellation

(76) Inventor: Paul D. Daly, Troy, MI (US)

Correspondence Address: LAURA M. SLENZAK, SIEMENS CORPORATION, 186 WOOD AVENUE SOUTH, ISELIN, NJ, 08830, US

(21) Appl. No.: 09/901,237

(22) Filed: Jul. 9, 2001

Related U.S. Application Data
(60) Provisional application No. 60/234,315, filed on Sep. 20, 2000.

(51) Int. Cl. A61F 11/06; G10K 11/16; H03B 29/00

(52) U.S. Cl. 381/71.4; 381/71.5; 381/86; 381/71.9

(57) ABSTRACT

The air induction system comprises an air induction body and speaker disposed about the air induction body. A control unit communicates with the speaker and has at least two modes of noise attenuation signal generation. An engine speed sensor communicates engine speed data to the control unit. An engine load sensor communicates engine load data to the control unit as well. The control unit then selects one of the at least two modes of noise attenuation based on the engine speed data and the engine load data.

This application claims priority to Provisional Patent Application Serial No. 60/234,315 filed Sep. 9, 2000.

### BACKGROUND OF THE INVENTION

This invention relates to an active method and system for controlling automotive induction noise.

Manufacturers have employed active and passive methods to reduce engine noise within the passenger compartment. Such noise frequently emanates from the engine, travels through the air induction system and emanates out of the mouth of the air intake into the passenger compartment. Efforts have been made to reduce the amount of engine noise traveling through the air induction system. These efforts include the use of both passive devices such as expansion chambers and Helmholtz resonators and active devices involving anti-noise generators.

Active systems use a speaker to create a canceling sound that attenuates engine noise. The sound created is out of phase with the engine noise and combines with this noise to result in its reduction. Generally, this sound is generated in proximity to the mouth of the air induction system. In one such system, a control unit, such as a digital signal processor, obtains data from the vehicle engine, creates a predictive model of engine noise, and thereby generates the appropriate cancellation signal based on the results of this model. This signal is then transmitted to the speaker, which transforms this signal into a canceling sound. Because the control unit may not perfectly model engine noise, an error microphone is placed in proximity to the mouth of the air induction system to determine if engine noise need be further attenuated.

Some cars have two modes of driving: a normal operation mode and a sport mode. Typically, a normal operation mode will allow an operator to drive the vehicle to maximize fuel economy while a sport mode will allow the operator to drive the vehicle for optimal power. A switch to the vehicle transmission permits the operator to alternate between the two modes of driving.

In sport mode, an operator generally prefers to hear the sound of the vehicle engine. However, an active noise attenuation system may eliminate this aural feedback, thereby preventing the vehicle engine from sounding sporty. Systems do exist that provide for two modes of noise attenuation signal generation: one mode for the normal mode operation of the vehicle and another mode for the sport mode operation of the vehicle. The normal mode of noise attenuation may attenuate engine noise to a higher degree than the sport mode of noise attenuation. These systems employ a hardwire connection between the noise attenuation system and the transmission of the vehicle so as to permit the noise attenuation system to track the switch between normal mode and sport mode. An additional connection between the transmission and the noise attenuation system is undesirable, adding complexity and expense to the noise attenuation system.

A need therefore exists for a simpler means of switching the noise attenuation system between the two modes of noise attenuation signal generation.

### SUMMARY OF THE INVENTION

The present invention offers the benefit of an uncomplicated noise attenuation solution for multiple mode operation of a vehicle transmission and engine. The air induction system comprises an air induction body and a speaker disposed about the air induction body. A control unit communicates with the speaker and has at least two modes of noise attenuation signal generation. One mode may be a mode for noise attenuation during normal driving operation while another mode may serve to attenuate noise for sport driving operation. Rather than employ a new hardwire connection between the engine transmission and the noise attenuation system, the current invention relies on existing components of noise attenuation systems to allow the control unit to select the proper mode of noise attenuation. Indeed, engine speed sensors and engine load sensors are already used in such systems. The control unit may be programmed so as to obtain engine speed data and engine load data from these sensors and select the proper mode of noise attenuation based on the detected data.

In one such embodiment, the air induction system includes a memory unit that stores driving mode information. This driving mode information serves to assist the control unit in the selection of one of the two modes of noise attenuation signal generation. The driving mode information may comprise data that ties engine speed data to a particular mode of driving and thus mode of noise attenuation. The information may also comprise data that ties engine load data to such a mode. Accordingly, when a particular condition of engine speed and engine load is met, the control unit commences noise attenuation in the mode associated with this particular condition. For example, if the control unit senses high engine speed and engine load, then the control unit may conclude that the operator is driving in sport mode and commence sport mode noise attenuation. Conversely, if the control unit senses low engine speed and low engine load, then the control unit may conclude that the operator is in normal driving mode and may commence normal mode noise attenuation. While one embodiment may have two modes of noise attenuation, such as one for normal driving and one for sport driving, the invention envisions that additional modes of noise attenuation may be selected in this way.

The method of noise attenuation embodies a straightforward manner of selecting between noise attenuation modes. Engine speed and engine load are determined. Then based on the determined engine speed data and engine load data, one of the two modes of noise attenuation is selected. A noise attenuation signal is then generated from the selected mode.

Again the modes may comprise a normal driving mode and a sport driving mode. The control unit simply selects between these modes by comparing the determined engine speed data and engine load data with engine speed data and engine load data related to each of two modes of noise attenuation signal generation. The relationship between the modes and the expected data would be known and could be easily stored. The present system and method thereby eliminates the need for a hardwire connection to the transmission. Instead, the system and method relies on currently used noise attenuation components to accomplish the task of selecting between modes of noise attenuation operation.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 illustrates a schematic embodiment of the invention, including air induction body, speaker, control unit, engine speed sensor, and engine load sensor.

FIG. 2 illustrates a flowchart diagram of an embodiment of the invention.

### DETAILED DESCRIPTION OF THE PREFERRED EMBODIMENT

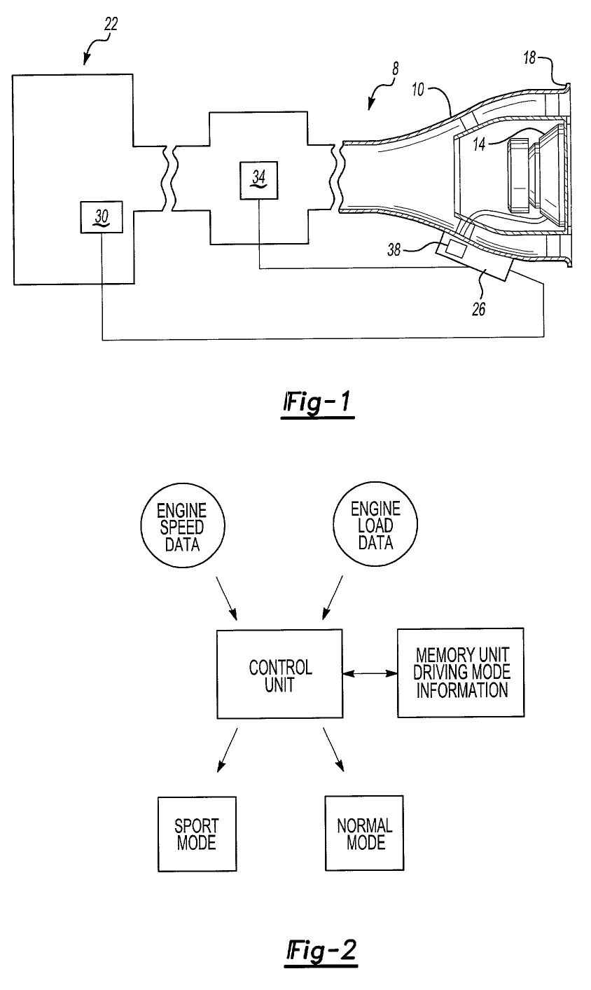

The invention is air induction system 8 that employs components commonly used in existing noise attenuation systems to select the proper mode of noise attenuation signal generation. As seen if FIG. 1, the system comprises an air induction body 10 and speaker 14 disposed about air induction body 10. Speaker 14 is shown in the same plane as mouth 18 of air induction body 10. While the speaker is shown mounted in a particular location, it is also contemplated that such noise attenuation speakers can be mounted at other locations such as in the vehicle, separate from body 10. All that is required is that the speaker be adjacent body 10. Noise emanates from engine 22 through air induction body 10 and out mouth 18. Control unit 26 communicates and controls speaker 14 to attenuate noise from engine 22. As known, control unit 26 generates a sound wave out of phase with noise from the engine to thereby cancel both sounds. Control unit 26 also communicates with engine speed sensor 30 and engine load sensor 34 and obtains engine speed data and engine load data to determine the type of attenuating sound wave to generate. Engine speed sensor 30 may be a tachometer while engine load sensor may be a mass air flow sensor or manifold absolute pressure sensor, which as known, provide data on the amount of load experienced by engine 22.

Unlike existing systems, control unit 26 has at least two modes of noise attenuation signal generation and uses engine load data and engine speed data to select the correct mode of operation. One mode of noise attenuation may relate to the generation of noise attenuation sounds for normal driving operation. As known, such noise attenuation may strive to eliminate all engine noise to provide a quiet interior. Another mode of noise attenuation may provide a lower degree of noise attenuation to permit more engine noise to travel back to the passenger compartment, thereby providing the operator with greater engine sound feedback. The selection of the mode of noise attenuation signal generation depends on the detected engine speed data and engine load data.

Memory unit 38 may store driving mode information that at least assists control unit 26 in the selection of one of the at least two modes of noise attenuation signal generation. Such driving mode information may be stored in memory unit 38 by vehicle manufacturer during vehicle assembly. Memory unit 38 communicates such information to control unit 26, which then uses this information to make the correction selection. Driving mode information may comprise stored data relating at least one mode of noise attenuation to engine speed data. For example, high engine speeds are typically associated with sport mode driving and suggest that sport mode noise attenuation may be required. Alternatively, low engine speeds may suggest normal mode operation. When engine speeds (such as RPM speeds) are high, then low, and then quickly high once more, this driving mode information suggests quick gear changes and once more sport driving. The opposite suggests normal driving. Accordingly, engine speed data may be tied to sport mode or to normal mode and consequently, when detected, may suggest the selection of one or the other mode of noise attenuation by control unit 26. Essentially, control unit 26 may monitor upshift speeds and kick down load points to determine whether the driver is operating in sport mode or normal mode. Quick upshifts and high engine loads suggest sport mode while slower upshifts and low engine loads suggest normal mode.

Engine load sensor 34 provides additional feedback to permit control unit 26 to make a mode selection. High engine loads suggests sport driving while low engine loads suggest normal vehicle operation. Hence, engine load data provides control unit 26 with driving mode information that may be used to select the proper mode of noise attenuation signal generation.

Preferably, air induction system 8 employs both engine load sensor 34 and engine speed sensor 30 to provide data to control unit 26. Memory unit 38 may accordingly store a table of engine speed data and engine load data associated with each mode of driving whether normal driving or sport driving. This embodiment provides greater accuracy in the selection of the proper mode of noise attenuation signal generation.

FIG. 2 illustrates a schematic diagram of an embodiment of the invention. Engine speed data and engine load data serve as inputs into control unit. As described above, memory unit interacts with control unit to assist control unit in the selection of the proper driving mode, either sport mode noise attenuation signal generation or normal mode noise attenuation signal generation. Once the proper mode is selected, then control unit outputs the appropriate noise attenuation sound, whether for normal driving or sport mode driving.

Memory unit 38 may also track and weigh the engine speed data and engine load data detected by engine speed sensor 30 and engine load sensor 34. Memory unit 38 may store this data in a table and weigh these values. Accordingly, memory unit 38 and control unit 26 could track the particular speed or load of engine 22, weigh these values based on the amount of time the engine 22 has maintained the particular speed or load, and then add these values to determine whether the vehicle is in sport mode or normal operation mode. The longer high loads and high speeds are maintained, the more the data would indicate the operation of the vehicle to be in sport mode. The opposite would suggest normal mode.

Memory unit 38 could be reset at predetermined intervals to permit control unit 26 to reevaluate the mode of noise attenuation. For example, memory unit 38 may be reset if engine speed or engine load is below a predetermined threshold for a predetermined amount of time. Also, memory unit 38 may reset when the engine is shut off. In this way, control unit 26 would not always operate in the same mode.

Hence, the invention encompasses a method of noise attenuation whereby engine speed data and engine load data are determined. One of at least two modes of noise attenuation signal generation is then selected based on the determined data. Once the correct mode is selected, whether normal operating mode or sport mode, a noise attenuation signal is generated based on the selected mode. If the mode is sport mode noise attenuation signal generation, then a noise attenuation signal that attenuates engine noise less is employed. On the other hand, if the mode of normal mode noise attenuation signal generation is selected, then more engine noise is thereby attenuated. The selection may be based on a comparison of determined engine speed data and engine load data with engine speed data and engine load data related to each of the modes of noise attenuation signal generation.

The aforementioned description is exemplary rather then limiting. Many modifications and variations of the present invention are possible in light of the above teachings. The preferred embodiments of this invention have been disclosed. However, one of ordinary skill in the art would recognize that certain modifications would come within the scope of this invention. Hence, within the scope of the appended claims, the invention may be practiced otherwise than as specifically described. For this reason the following claims should be studied to determine the true scope and content of this invention.

What is claimed is:

1. An air induction system comprising:
an air induction body;
a speaker;
a control unit in communication with said speaker, having at least two modes of noise attenuation signal generation;
an engine sensor communicating engine data to said control unit; and
said control unit selecting one of said at least two modes of noise attenuation signal generation based on said engine data.

2. The air induction system of claim 1 where said engine data comprises engine load data and engine speed data.

3. The air induction system of claim 1 including a memory unit storing driving mode information that at least assists said control unit in the selection of one of said at least two modes of noise attenuation signal generation.

4. The air induction system of claim 3 wherein said driving mode information comprises data relating at least one mode of noise attenuation to said engine speed data.

5. The air induction system of claim 3 wherein said driving mode information comprises data relating at least one mode of noise attenuation to said engine load data.

6. The air induction system of claim 3 wherein said driving mode information comprises data relating at least one mode of noise attenuation to said engine load data and said engine speed data.

7. The air induction system of claim 1 wherein one of said at least two driving modes comprises a sport-driving mode and one of said at least two driving modes comprises a normal driving mode.

8. An air induction system comprising:
an air induction body;
a speaker disposed adjacent said air induction body;
a control unit in communication with said speaker, having at least two modes of noise attenuation signal generation;
a memory unit storing driving mode information that assists said control unit in the selection of one of said at least two modes of noise attenuation signal generation;
an engine speed sensor communicating engine speed data to said control unit; and
an engine load sensor communicating engine load data to said control unit wherein said control unit selects one of said at least two modes of noise attenuation signal generation based on a comparison of said engine speed data and said engine load data and data stored in said memory unit.

9. The air induction system of claim 8 wherein said driving mode information comprises data relating at least one mode of noise attenuation to said engine speed data.

10. The air induction system of claim 9 wherein said driving mode information comprises data relating at least one mode of noise attenuation to said engine load data.

11. The air induction system of claim 9 wherein said driving mode information comprises data relating at least one mode of noise attenuation to said engine load data and said engine speed data.

12. The air induction system of claim 8 wherein one of said at least two driving modes comprises a sport-driving mode and one of said at least two driving modes comprises a normal driving mode.

13. A method of noise attenuation comprising:
determining engine speed data;
determining engine load data;
selecting one of at least two modes of noise attenuation signal generation based on the determined engine speed data and engine load data; and
generating a noise attenuation signal from the selected mode.

14. The method of claim 13 wherein one of the at least two driving modes comprises a sport-driving mode.

15. The method of claim 13 wherein one of the at least two driving modes comprises a normal driving mode.

16. The method of claim 13 wherein one of the at least two driving modes comprises a sport-driving mode and one of the at least two driving modes comprises a normal driving mode.

17. The method of claim 13 wherein the selecting one of at least two modes of noise attenuation signal generation comprises comparing the determined engine speed data and engine load data with engine speed data and engine load data related to each of the at least two modes of noise attenuation signal generation.
